(19) United States
(12) Patent Application Publication
(10) Pub. No.: US 2025/0361256 A1
(43) Pub. Date: Nov. 27, 2025

(54) SEPARATION OF HUMAN MILK OLIGOSACCHARIDES FROM A FERMENTATION BROTH

(71) Applicant: DSM IP ASSETS B.V., Maastricht (NL)

(72) Inventors: Nikolay KHANZHIN, Humlebæk (DK); Egle PAKALNYTE, Copenhagen (DK)

(21) Appl. No.: 18/874,209

(22) PCT Filed: Jun. 13, 2023
(86) PCT No.: PCT/EP2023/065810

§ 371 Date: Dec. 12, 2024

(30) Foreign Application Priority Data
Jun. 14, 2022 (DK) PA202200566
Jun. 14, 2022 (DK) PA202200567

(51) Int. Cl. C07H 3/06; B01D 61/02; B01D 61/14; B01D 61/58; B01D 71/56; B01J 20/26; B01J 20/28; C07H 1/08

(52) U.S. Cl. CPC C07H 3/06; B01D 61/027; B01D 61/145; B01D 61/58; B01D 71/56; B01J 20/267; B01J 20/28061; B01J 20/28064; B01J 20/28066; C07H 1/08; B01D 2311/2626

(57) ABSTRACT

The invention relates to a method for the purification of a neutral or sialylated human milk oligosaccharide (HMO) from a fermentation broth. Moreover, the invention also concerns neutral or sialylated HMOs obtained by the inventive method, as well as its use in food, feed, and medical application.

### FIELD OF THE INVENTION

The present invention relates to the separation and isolation of neutral or sialylated human milk oligosaccharides (HMOs) from a reaction mixture in which they are produced.

### BACKGROUND OF THE INVENTION

During the past decades, the interest in the preparation and commercialisation of human milk oligosaccharides (HMOs) has been increasing steadily. The importance of HMOs is directly linked to their unique biological activities. Therefore, HMOs have become important potential products for nutrition and therapeutic uses. As a result, low cost ways of producing industrially HMOs have been sought.

To date, the structures of more than 140 HMOs have been determined, and considerably more are probably present in human milk (Urashima et al.: Milk oligosaccharides, Nova Biomedical Books, 2011; Chen Adv. Carbohydr. Chem. Biochem. 72, 113 (2015)). The HMOs comprise a lactose (Galβ1-4Glc) moiety at the reducing end and may be elongated with an N-acetylglucosamine, or one or more N-acetyllactosamine moiety/moieties (Galβ1-4GlcNAc) and/or a lacto-N-biose moiety (Galβ1-3GlcNAc). Lactose and the N-acetyllactosaminylated or lacto-N-biosylated lactose derivatives may further be substituted with one or more fucose and/or sialic acid residue(s), or lactose may be substituted with an additional galactose, to produce HMOs known so far.

Direct fermentative production of HMOs, especially of those being a trisaccharide, has recently become practical (Han et al. Biotechnol. Adv. 30, 1268 (2012) and references cited therein). Such fermentation technology has used a recombinant E. coli system wherein one or more types of glycosyl transferases originating from viruses or bacteria have been co-expressed to glycosylate exogenously added lactose, which has been internalized by the LacY permease of the E. coli. However, the use of a recombinant glycosyl transferase, especially series of recombinant glycosyl transferases to produce oligosaccharides of four or more monosaccharide units, has always led to by-product formation hence resulting in a complex mixture of oligosaccharides in the fermentation broth. Further, a fermentation broth inevitably contains a wide range of non-carbohydrate substances such as cells, cell fragments, proteins, protein fragments, DNA, DNA fragments, endotoxins, caramelized by-products, minerals, salts, or other charged molecules.

For separating HMOs from carbohydrate by-products and other contaminating components, active carbon treatment combined with gel filtration chromatography has been proposed as a method of choice (WO 01/04341, EP-A-2479263, Dumon et al. Glycoconj. J. 18, 465 (2001), Priem et al. Glycobiology 12, 235 (2002), Drouillard et al. Angew. Chem. Int. Ed. 45, 1778 (2006), Gebus et al. Carbohydr. Res. 361, 83 (2012), Baumgärtner et al. ChemBioChem 15, 1896 (2014)). Although gel filtration chromatography is a convenient lab scale method, it cannot be efficiently scaled up for industrial production. Instead, recent methods comprise ion exchange resin treatment for removing charged organic and inorganic substances combined with active charcoal treatment for decolorization (see e.g. WO 2015/106943, WO 2017/182965). However, active carbon treatment generally has the disadvantage that active carbon has the potential to adsorb relatively high amounts of HMOs, which may lead to decreased HMO yields. Moreover, the regeneration of active carbon is complicated, which makes the reuse of active carbon less attractive.

Alternative and/or improved procedures for isolating and purifying neutral or sialylated HMOs from non-carbohydrate components of the fermentation broth in which they have been produced, especially those suitable for industrial scale, are needed to improve the recovery yield of neutral or sialylated HMOs and/or to simplify prior art methods while the purity of the neutral or sialylated HMOs is at least maintained, and preferably, improved. Moreover, such alternative purification procedures preferably lead to purified neutral or sialylated HMOs that are free of proteins and recombinant materials originating from the used recombinant microbial strains, which are thus well suited for use in food, medical food, and feed applications.

### SUMMARY OF THE INVENTION

The invention relates to a method for the purification of a neutral or sialylated human milk oligosaccharides (HMOs) from a fermentation broth, comprising the steps of:
I. separating an HMO-containing stream from biomass;
II. purifying the HMO-containing stream with an adsorbent resin, and
III. concentrating and drying the purified HMO-containing stream to obtain the neutral or sialylated HMOs in solidified form.

Preferably, the method does not comprise treatment with ion exchange resin.

Also preferably, purification/decolourization step with active carbon is excluded.

In one embodiment, purification with ultrafiltration or nanofiltration is performed between steps I) and II).

In one embodiment, purification with ultrafiltration and/or nanofiltration is performed between steps II) and III).

In one embodiment, demineralization with electrodialysis is performed between steps II) and III).

In another aspect, the invention relates to neutral or sialylated human milk oligosaccharides obtained by the method according to the invention.

Another aspect of the invention relates to neutral or sialylated human milk oligosaccharides obtained by the method according to the invention for use in medicine.

Another aspect of the invention relates to the use of neutral or sialylated human milk oligosaccharides obtained by the method according to the invention for food and/or feed applications.

Another aspect of the invention relates to a food or cosmetic product comprising neutral or sialylated human milk oligosaccharides obtained by the method according to the invention.

### DETAILED DESCRIPTION OF THE INVENTION

### 1. Terms and Definitions

The term “fermentation broth”, as used in this specification, refers to a product obtained from fermentation of the microbial organism. Thus, the fermentation product comprises cells (biomass), the fermentation medium, salts, residual substrate material, and any molecules/by-products produced during fermentation, such as the desired neutral or sialylated HMOs. After each step of the purification method, one or more of the components of the fermentation product is removed, resulting in more purified neutral or sialylated HMOs.

The term “monosaccharide” means a sugar of 5-9 carbon atoms that is an aldose (e.g. D-glucose, D-galactose, D-mannose, D-ribose, D-arabinose, L-arabinose, D-xylose, etc.), a ketose (e.g. D-fructose, D-sorbose, D-tagatose, etc.), a deoxysugar (e.g. L-rhamnose, L-fucose, etc.), a deoxy-aminosugar (e.g. N-acetylglucosamine, N-acetylmannosamine, N-acetylgalactosamine, etc.), a uronic acid, a ketoaldonic acid (e.g. sialic acid) or equivalents.

The term “disaccharide” means a carbohydrate consisting of two monosaccharide units linked to each other by an interglycosidic linkage.

The term “tri- or higher oligosaccharide” means a sugar polymer consisting of at least three, preferably from three to eight, more preferably from three to six, monosaccharide units (vide supra). The oligosaccharide can have a linear or branched structure containing monosaccharide units that are linked to each other by interglycosidic linkages.

The term “human milk oligosaccharide” or “HMO” means a complex carbohydrate found in human breast milk (Urashima et al.: Milk Oligosaccharides, Nova Medical Books, NY, 2011; Chen Adv. Carbohydr. Chem. Biochem. 72, 113 (2015)). The HMOs have a core structure being a lactose unit at the reducing end that is elongated i) by a β-N-acetyl-glucosaminyl group or ii) by one or more β-N-acetyl-lactosaminyl and/or one or more β-lacto-N-biosyl units, and which core structures can be substituted by an α-L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose II (LNTri, GlcNAc(β1-3)Gal(β1-4)Glc), lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

The term “sialyl” or “sialyl moiety” means the glycosyl residue of sialic acid (N-acetyl-neuraminic acid, Neu5 Ac), preferably linked with α-linkage:

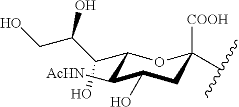

The term “fucosyl” means an L-fucopyranosyl group, preferably linked with α-interglycosidic linkage:

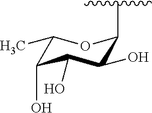

“N-acetyl-glucosaminyl” means an N-acetyl-2-amino-2-deoxy-D-glucopyranosyl (GlcNAc) group, preferably linked with β-linkage:

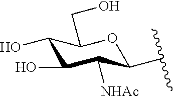

“N-acetyl-lactosaminyl” means the glycosyl residue of N-acetyl-lactosamine (LacNAc, Galpβ1-4GlcNAc), preferably linked with β-linkage:

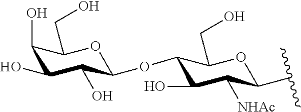

Furthermore, the term “lacto-N-biosyl” means the glycosyl residue of lacto-N-biose (LNB, Galpβ1-3GlcNAc), preferably linked with β-linkage:

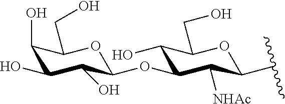

The term “biomass”, in the context of fermentation, refers to the suspended, precipitated, or insoluble materials originating from fermentation cells, like intact cells, disrupted cells, cell fragments, proteins, protein fragments, polysaccharides.

The term “Brix” refers to degrees Brix, that is the sugar content of an aqueous solution (g of sugar in 100 g of solution). In this regard, Brix of the human milk oligosaccharide solution of this application refers to the overall carbohydrate content of the solution including the human milk oligosaccharides and its accompanying carbohydrates. Brix is measured by a calibrated refractometer.

“Demineralization” preferably means a process of removing minerals or mineral salts from a liquid. In the context of the present invention, demineralization can occur in the nanofiltration step, especially when it is combined with diafiltration, or by using cation and anion exchange resins (if applicable).

The term “protein-free aqueous medium” preferably means an aqueous medium or broth from a fermentation or enzymatic process, which has been treated to remove substantially all the proteins, as well as peptides, peptide fragments, RNAs and DNAs, as well as endotoxins and glycolipids that could interfere with the eventual purification of the one or more neutral or sialylated HMOs and/or one or more of their components, especially the mixture thereof, from the fermentation or enzymatic process mixture.

The term “HMO-containing stream” means an aqueous medium containing neutral or sialylated HMOs obtained from a fermentation process, which has been treated to remove suspended particulates and contaminants from the process, particularly cells, cell components, insoluble metabolites and debris that could interfere with the eventual purification of the one or more hydrophilic oligosaccharides, especially one or more neutral or sialylated HMOs and/or one or more HMO components, especially mixtures thereof.

The term “biomass waste stream” preferably means suspended particulates and contaminants from the fermentation process, particularly cells, cell components, insoluble metabolites, and debris.

Rejection factor of a salt (in percent) is calculated as (1−κₚ/κᵣ)·100, wherein κₚ is the conductivity of the salt in the permeate and κᵣ is the conductivity of the salt in the retentate.

Rejection factor of a carbohydrate (in percent) is calculated as (1−Cₚ/Cᵣ)·100, wherein Cₚ is the concentration of the carbohydrate in the permeate and Cᵣ is the concentration of the carbohydrate in the retentate.

The term “diafiltration” refers to solvent addition (water) during the membrane filtration process. If diafiltration is applied during ultrafiltration, it improves the yield of the desired HMO in the permeate. If diafiltration is applied during nanofiltration, it improves the separation of small size impurities and salts to the permeate. The solute yield and therefore the product enrichment could be calculated based on the formulas known to the skilled person based on rejection factors and relative amount of water added.

The term “concentrating” refers to the removal of liquid, mostly water, thus resulting in a higher concentration of the neutral or sialylated HMOs in the purified HMO-containing product stream.

The term “decolorization” refers to the process of removing colour bodies from a solution to the extent required by product specifications. The decolorization of carbohydrate-containing solutions is mainly based on Van-der-Waals type interactions of the colour bodies with the adsorbent. In the context of the present invention, the colour of the solution is quantified by absorption of visible light at 400 nm (Abs_400) and normalized by the concentration and the path length. Thereby, the colour index CI 400 is defined as 1000×Abs_400/Brix with the path length=1 cm. Normally, if the CI_400<5, then a solid product isolated from its solution appears as colourless (white) solid. A crude supernatant solution containing HMO product after fermentation usually has a colour index CI 400 in the range from 100 to 400.

### 2. Method for the Purification of a Neutral or Sialylated HMO from a Fermentation Broth

The invention relates to a method for the purification of a neutral or sialylated human milk oligosaccharide (HMO) from a fermentation broth, comprising the steps of:
I. separating an HMO-containing stream from biomass;
II. purifying the HMO-containing stream with an adsorbent resin, and
III. concentrating and drying the purified HMO-containing stream to obtain the neutral or sialylated HMO in solidified form.

### The Fermentation Broth

In an embodiment, the neutral or sialylated HMO being present in the fermentation broth has been obtained by culturing a genetically modified microorganism capable of producing said neutral or sialylated human milk oligosaccharide from an internalized carbohydrate precursor. Preferably, the microbial organism is a genetically modified bacterium or yeast such as a Saccharomyces strain, a Candida strain, a Hansenula strain, a Kluyveromyces strain, a Pichia strain, a Schizosaccharomyces stain, a Schwanniomyces strain, a Torulaspora strain, a Yarrowia strain, or a Zygosaccharomyces strain. More preferably, the yeast is Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, or Zygosaccharomyces bailii; and the Bacillus is Bacillus amyloliquefaciens, Bacillus licheniformis or Bacillus subtilis.

In an embodiment, at least one neutral or sialylated human milk oligosaccharide being present in the fermentation broth has not been obtained by microbial fermentation, but has been e.g. added to the fermentation broth after it has been produced by a non-microbial method, e.g. chemical and/or enzymatic synthesis.

In an embodiment, the purity of the neutral or sialylated HMO in the fermentation broth is ≤70%, preferably ≤60%, more preferably ≤50%, most preferably ≤40%.

Preferably, the HMO is a neutral HMO. In an embodiment, the neutral HMO is preferably selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-neofucopentaose V (alternative name: lacto-N-fucopentaose VI), lacto-N-difucohexaose I, lacto-N-difucohexaose II, lacto-N-difucohexaose III, 6′-galactosyllactose, 3′-galactosyllactose, lacto-N-hexaose, lacto-N-neohexaose, and any mixture thereof. More preferably, the HMO is 2′-fucosyllactose, 3-fucosyllactose, 2′,3-difucosyllactose, lacto-N-triose II, lacto-N-tetraose, lacto-N-neotetraose or a lacto-N-fucopentaose, more preferably 2′-fucosyllactose, 3-fucosyllactose or 2′,3-difucosyllactose.

In an embodiment, the sialylated HMO is selected from the group consisting of 3′-sialyllactose (3′-SL) and 6′-sialyllactose (6′-SL).

In an embodiment, the HMO in the fermentation broth is a single neutral or sialylated HMO.

In an embodiment, the HMO in the fermentation broth is a mixture of various individual neutral or sialylated HMOs.

In an embodiment, the HMO is a mixture of two individual neutral or sialylated HMOs. In another embodiment, the HMO is a mixture of three individual neutral or sialylated HMOs. In another embodiment, the HMO is a mixture of four individual neutral or sialylated HMOs. In another embodiment, the HMO is a mixture of five individual neutral or sialylated HMOs.

In an embodiment, the HMO in the fermentation broth is a mixture of a neutral or sialylated HMO obtained by microbial fermentation and a HMO that has not been obtained by microbial fermentation, but e.g. by chemical and/or enzymatic synthesis.

### Separating the Fermentation Broth to Form an HMO-Containing Stream and a Biomass Waste Stream in Step I) of the Method According to the Invention

In step I) of the method according to the invention, the HMO-containing stream is separated from the biomass waste stream.

The fermentation broth typically contains, besides the desired neutral or sialylated HMO, the biomass of the cells of the used microorganism together with proteins, protein fragments, peptides, DNAs, RNAs, endotoxins, biogenic amines, amino acids, organic acids, inorganic salts, unreacted carbohydrate acceptors such as lactose, sugar-like by-products, monosaccharides, colorizing bodies, etc. In step I) of the method according to the invention, the biomass is separated from the neutral or sialylated HMO.

In a preferred embodiment, the biomass is separated from the neutral or sialylated HMO in step I) by ultrafiltration. The ultrafiltration step is to separate the biomass and, preferably, also high molecular weight components and suspended solids from the lower molecular weight soluble components of the broth, which pass through the ultrafiltration membrane in the permeate. This ultrafiltration permeate is an aqueous solution containing the neutral or sialylated human milk oligosaccharide also referred to as the HMO-containing stream, whereas the ultrafiltration retentate comprises the biomass waste stream.

Any conventional ultrafiltration membrane can be used having a molecular weight cut-off (MWCO) range between about 1 and about 500 kDa, such as 10-250, 50-100, 200-500, 100-250, 1-100, 1-50, 10-25, 1-5 kDa, or any other suitable sub-range. The membrane material can be a ceramic or made of a synthetic or natural polymer, e.g. polysulfone, polyvinylidene fluoride, polyacrylonitrile, polypropylene, cellulose, cellulose acetate or polylactic acid. The ultrafiltration step can be applied in dead-end or cross-flow mode. Step I) of the method according to the invention may comprise more than one ultrafiltration step using membranes with different MWCO as defined above, e.g. applying two ultrafiltration separations, wherein the first membrane has a higher MWCO than that of the second membrane. This arrangement may provide a better separation efficacy of the higher molecular weight components of the broth. After this separation step, the permeate contains materials that have a molecular weight lower than the MWCO of the second membrane, including the neutral or sialylated human milk oligosaccharides of interest.

In one embodiment, the fermentation broth is ultrafiltered using a membrane having a MWCO of 5 to 30 kDa, such as 10-25, 15 or 20 kDa.

In a preferred embodiment, the yield of the desired neutral or sialylated HMO in the permeate after the ultrafiltration step performed in step I) is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In another embodiment, the broth obtained from fermentation is subjected to centrifugation to separate the biomass from the neutral or sialylated HMO (HMO-containing stream) in step I) of the method according to the invention. In said embodiment, the supernatant represents the HMO-containing stream, while the remaining material, i.e. the “biomass waste stream” can be separated out. By centrifugation, a clear supernatant comprising the neutral or sialylated HMO can be obtained, which represents the HMO-containing stream.

The centrifuging can be lab scale or, advantageously over previous centrifuging methods, commercial scale (e.g. industrial scale, full production scale).

In some embodiments, a multi-step centrifugation can be used. For example, a series of 2, 3, 4, 5, 6, 7, 8, 9, or 10 centrifugation steps can be performed. In other embodiments, the centrifugation may be a single step. Centrifugation provides for a quick biomass-removal.

In certain embodiments, Sedicanter® centrifuge designed and manufactured by Flottweg can be used.

The particular type of centrifuge is not limiting, and many types of centrifuges can be used. The centrifuging can be a continuous process. In some embodiments, the centrifuging can have feed addition. For example, the centrifuging can have a continuous feed addition. In certain embodiments, the centrifuging can include a solid removal, such as a wet solid removal. The wet solid removal can be continuous in some implementations, and periodic in other implementations.

For example, a conical plate centrifuge (e.g. disk bowl centrifuge or disc stack separator) can be used. The conical plate centrifuge can be used to remove solids (usually impurities) from liquids, or to separate two liquid phases from each other by means of a high centrifugal force. The denser solids or liquids which are subjected to these forces move outwards towards the rotating bowl wall while the less dense fluids move towards the centre. The special plates (known as disc stacks) increase the surface settling area which speeds up the separation process. Different stack designs, arrangements and shapes are used for different processes depending on the type of feed present. The concentrated denser solid or liquid can then be removed continuously, manually, or intermittently, depending on the design of the conical plate centrifuge. This centrifuge is very suitable for clarifying liquids that have small proportion of suspended solids.

The centrifuge works by using the inclined plate setter principle. A set of parallel plates with a tilt angle θ with respect to horizontal plane is installed to reduce the distance of the particle settling. The reason for the tilted angle is to allow the settled solids on the plates to slide down by centrifugal force so they do not accumulate and clog the channel formed between adjacent plates.

This type of centrifuge can come in different designs, such as nozzle-type, manual-cleaning, self-cleaning, and hermetic. The particular centrifuge is not limiting.

Factors affecting the centrifuge include disk angle, effect of g-force, disk spacing, feed solids, cone angle for discharge, discharge frequency, and liquid discharge.

Alternatively, a solid bowl centrifuge (e.g. a decanter centrifuge) can be used. This is a type of centrifuge that uses the principle of sedimentation. A centrifuge is used to separate a mixture that consists of two substances with different densities by using the centrifugal force resulting from continuous rotation. It is normally used to separate solid-liquid, liquid-liquid, and solid-solid mixtures. One advantage of solid bowl centrifuges for industrial uses is the simplicity of installation compared to other types of centrifuge. There are three design types of solid bowl centrifuge, which are conical, cylindrical, and conical-cylindrical.

Solid bowl centrifuges can have a number of different designs, any of which can be used for the disclosed method. For example, conical solid bowl centrifuges, cylindrical solid bowl centrifuges, and conical-cylindrical bowl centrifuges can be used.

The centrifuging can be performed at a number of speeds and residence times. For example, the centrifuging can be performed with a relative centrifugal force (RCF) of 20000 g, 15000 g, 10000 g, or 5000 g. In some embodiments, the centrifuging can be performed with a relative centrifugal force (RCF) of less than 20000 g, 15000 g, 10000 g or 5000 g. In some embodiments, the centrifuging can be performed with a relative centrifugal force (RCF) of greater than 20000 g, 15000 g, 10000 g or 5000 g.

In some embodiments, the centrifuging can be characterized by working volume. In some embodiments, the working volume can be 1, 5, 10, 15, 20, 50, 100, 300, or 500 l. In some embodiments, the working volume can be less than 1, 5, 10, 15, 20, 50, 100, 300, or 500 l. In some embodiments, the working volume can be greater than 1, 5, 10, 15, 20, 50, 100, 300, or 500 l.

In some embodiments, the centrifuging can be characterized by feed flow rate. In some embodiments, the feed flow rate can be 100, 500, 1000, 1500, 2000, 5000, 10000, 20000, 40000, or 100000 l/hr. In some embodiments, the feed flow rate can be greater than 100, 500, 1000, 1500, 2000, 5000, 10000, 20000, 40000, or 100000 l/hr. In some embodiments, the feed flow rate can be less than 100, 500, 1000, 1500, 2000, 5000, 10000, 20000, 40000, or 100000 l/hr.

The amount of time spent centrifuging (e.g. residence time) can vary as well. For example, the residence time can be 0.1, 0.2, 0.5, 1, 2, 3, 4, 5, 6, 7, 8, 9, or 10 minutes. In some embodiments, the residence time can be greater than 0.1, 0.2, 0.5, 1, 2, 3, 4, 5, 6, 7, 8, 9, or 10 minutes. In some embodiments, the residence time can be less than 0.1, 0.2, 0.5, 1, 2, 3, 4, 5, 6, 7, 8, 9, or 10 minutes.

Any of the above supernatant properties can be produced through a single instance of centrifuging. Alternatively, it can be produced through multiple instances of centrifuging.

In view of the above, step I) of the method according to the invention can be performed via ultrafiltration as defined above or centrifugation, or via a combination of ultrafiltration and centrifugation. Preferably, method step I) is carried out by ultrafiltration as defined above to obtain the HMO-containing stream separate from the biomass waste stream.

Before the ultrafiltration and/or centrifugation step, the fermentation broth can be subjected to a pre-treatment step. Pre-treatment of the fermentation broth can include pH adjustment, and/or dilution, and/or heat treatment. In certain implementations, all three of pH adjustment, dilution, and heat treatment can be performed. In alternative embodiments, pH adjustment and dilution can be performed. In alternative embodiments, pH adjustment and heat treating can be performed. In alternative embodiments, heat treating and dilution can be performed. Advantageously, a combination of a plurality of pre-treatment methods can provide an improved synergistic effect not found in individual pre-treatments.

In certain embodiments, one or more of the aforementioned pre-treatment steps can occur during the biomass removal in step I) by centrifuging and/or ultrafiltration as defined above. For example, between steps in a multi-step centrifuging, or the centrifuging vessel may be able to heat the fermentation broth during centrifuging.

Advantageously, the pre-treatment can increase the settling velocity of the solid particles (biomass) in the fermentation broth by a factor of 100 to 20000, making the biomass separation by centrifugation much more efficient and thus applicable in industrial scale. In addition to settling velocity, at least three other parameters are substantially improved due to pre-treatment that are, improved neutral or sialylated HMO yield in the HMO-containing stream, reduced protein and DNA content in the supernatant, and further residual suspended solid content can be substantially reduced.

Purifying the HMO-Containing Stream in Step II) with an Adsorbent Resin:

“Adsorbent” is an insoluble solid porous material with high specific surface area, i.e. >400 m²/g (as determined using the nitrogen sorption BET technique) capable of removing certain compounds from solution by physical adsorption and/or chemical sorption, whereas adsorption occurs on the pore surface of the adsorbent. Physical adsorption involves non-stoichiometric process with non-covalent interaction comprising of e.g. van der Waals, polar or ionic interactions without ion exchange. Chemical sorption or chemisorption proceeds due to a chemical reaction, i.e. involving electron exchange with chemical bond formation on the surface of the adsorbent. Examples of adsorbents are activated carbon (activated charcoal or AC) and adsorbent resins.

“Adsorbent resin” or “synthetic adsorbent” is specially designed macroporous polymer usually provided in spherical bed form with high specific surface area, e.g. >400 m²/g (as determined using the nitrogen sorption BET technique) and defined pore structure for selective removal of specific substances from an aqueous solution. In the context of the present application, the adsorbent resin suitable in step II) is partially functionalized with hydrophilic groups for easier regeneration. The adsorbent resin is therefore an acid adsorbent derived from cross-linked polystyrene or polyacrylic polymers and partially functionalized with primary, secondary or tertiary amines as weak base. In a preferred embodiment, the adsorbent resin used in step II) is a polystyrene-divinylbenzene copolymer with tertiary amine functional groups.

In the context of the present invention adsorbent resins are distinguished from ion exchangers or ion-exchange resins by a non-stoichiometric physical adsorption or by chemical adsorption such as acid adsorption by amines on polymeric support, where in both cases the molecules are removed from the feed solution as whole entities without releasing of ions or other substances from the adsorbent. In addition, an adsorbent resin functionalized with basic amino groups in free-base form does not contain any exchangeable ions and therefore could not be an ion exchanger if used in the free-base form. Also these resins usually have low basic capacity such as 0.1-0.3 eq/l (or around 0.6-1 eq/kg on dry weight), which is substantially below the typical capacity of an ion exchange resin, i.e. 1-2 eq/l. A resin capacity is a measurement of total capacity, as determined by a test performed in the lab by a titration methodology. A measured quantity of a basic (anion) resin, including a polystyrene-divinylbenzene copolymer adsorbent resin with tertiary amine functional groups used in step II), is fully converted to the basic form with an excess of strong base, e.g. NaOH-solution, and then well rinsed. A measured quantity of a strong inorganic acid, e.g. HCl or sulphuric acid solution, is then passed through the resin so as to totally exhaust the resin. The effluent is captured. The acid that passed through the column represents the amount that was not captured by the resin. This solution is then titrated with base to neutralize it and the amount of base required is expressed in equivalents. The difference between the total equivalents of acid passed through the column and the acid exiting the column represents the total equivalents of acid captured by the resin. The capacity of the resin is then determined.

In a preferred embodiment, the adsorbent resin has a surface area of >400 m²/g. In an embodiment, the adsorbent resin has a surface of from 400 to 1200 m²/g. More preferably, the adsorbent resin has a basic capacity of 0.1-0.3 eq/l (or around 0.6-1.0 eq/kg on dry weight). Further preferably, the adsorbent resin used in step II) is a polyacrylic, more preferably a methacrylic polymer adsorbent resin. Advantageously, the polymer is functionalized polystyrene (PS), more preferably cross-linked PS with divinylbenzene (DVB).

Suitable adsorbent resins are Dowex Optipore SD-2, Purolite MN100 or MN102.

In the context of the present invention, the substances to be removed are numerous coloured compounds, hydrophobic impurities, large biomolecules such as proteins, DNAs, RNAs, polysaccharides and their fragments.

In a preferred embodiment, purification with the adsorbent resin leads to an HMO-containing stream having a CI_400<5, preferably CI_400<4.

In a preferred embodiment, step II) results in a purified solution containing the HMO at a purity of ≥80%, preferably ≥85%, more preferably ≥90%.

The above defined adsorbent resin, in the method according to the invention, can be used as a replacement of activated carbon that is frequently used in known methods for purifying HMO streams. The adsorbent resin is as efficient as activated charcoal to remove colour, large biomolecules (proteins, DNAs etc.) and hydrophobic compounds, and has the advantages over the use of active carbon, that
it does not adsorb HMOs unlike charcoal. Charcoal may adsorb HMOs up to 30-35 wt % in relation of the weight of the charcoal used, which reduces the HMO recovery yield;
it could be easily regenerated and thus re-used many times like the active charcoal, where regeneration of charcoal requires harsh conditions or the use of organic solvents;
due to regeneration and lack of product adsorption, the amount of the adsorbent resin, unlike that of charcoal, is not limited, i.e. it could be used in any amount as long as it is sufficient to remove the impurities;
it lacks trace toxic elements such as nickel, lead and arsenic that can be present in the active carbon and that could be released into the product stream; and
due to the presence of the weakly basic sites, it is used as an insoluble base with acid adsorbent property, and thus replaces the active carbon together with the basic anion exchange resin treatment step that characterize the prior art methods.

### Concentrating and Drying the Purified HMO-Containing Stream to Obtain the Neutral or Sialylated HMO in Solidified Form in Step III):

The neutral or sialylated HMO of interest are provided in its solid form via a concentration and drying step (step III)).

A concentration step is used to economically remove significant quantities of liquid, mostly water, from the neutral or sialylated HMO-containing stream using e.g. evaporation, nanofiltration, or reverse-osmosis filtration. Evaporation processes can include, e.g. falling film evaporation, climbing film evaporation and rotary evaporation. The evaporation can also be conducted under vacuum. The incoming solids concentration to the process is preferably approximately 5 to 30 wt. %. The exit solids concentration from such a process is typically greater than 30 wt. %., preferably greater than 50 wt. %. More preferably, the solids concentration exiting the dewatering operation is 60 to 80 wt. %. The solids portion of the recovered material is preferably greater than 80 wt. % of neutral or sialylated HMO.

In an embodiment, the purified neutral or sialylated HMO-containing stream is concentrated to a concentration of ≥100 g/l of neutral or sialylated HMO, preferably of ≥200 g/l, more preferably of ≥300 g/l.

When the purified neutral or sialylated HMO-containing stream is concentrated by evaporation, the evaporation is preferably carried out at a temperature of from about 20 to about 80° C. In some embodiments, the evaporation is carried out at a temperature of from 25 to 75° C. In some embodiments, the evaporation is carried out at a temperature of from 30 to 70° C. In some embodiments, the evaporation is carried out at a temperature of from 30 to 65° C. Preferably, the evaporation is carried out under vacuum.

When the purified neutral or sialylated HMO-containing stream is concentrated by membrane filtration, any membrane, typically nanofiltration membrane, is suitable that sufficiently rejects the neutral or sialylated HMO. Concentration by membrane filtration usually provides an HMO-solution of around 30-35 wt %. This concentration may be suitable for conducting the subsequent drying-solidification step, e.g. freeze-drying. However, other drying methods may require more concentrated solutions, e.g. spray-drying or crystallization. In this case, concentration by evaporation, preferably under vacuum, is the preferred embodiment. Alternatively, the neutral or sialylated HMO-containing stream obtained in the previous step is concentrated to around 30-35 wt % using a nanofiltration membrane, and the solution is further concentrated by evaporation.

In one embodiment of the concentration by membrane filtration, the membrane of choice is a “tight” NF with 150-300 Da MWCO.

In other embodiment of the concentration by membrane filtration, the membrane of choice is a nanofiltration membrane that has a molecular weight cut-off (MWCO) of 500-3500 Da and an active (top) layer of polyamide (“loose” NF membrane); and the concentration step is performed so that the pH is set below 5.0, preferably below 4.5, advantageously below 4.0, but preferably not less than 3.0. In this latter embodiment, a substantial reduction of all monovalent salts in the retentate is also achievable. In said embodiment, the membrane is preferably a thin-film composite (TFC) membrane which is a piperazine-based polyamide membrane, more preferably its MgSO₄ rejection is about 50-90%, even more preferably its NaCl rejection is not more than 50%. An example of such a membrane is TriSep® UA60. Under this condition, remaining salts are also effectively removed, giving rise to a low-salt or a practically salt-free purified neutral or sialylated HMO-concentrate. In this embodiment, after completing the concentration step, the pH of the neutral or sialylated HMO-concentrate is advantageously set between 4-6 before performing the next step (e.g. evaporation, drying-solidification, sterile filtration).

The concentration step may be optional when the drying step is freeze-drying.

In an embodiment, the method according to the invention further comprises a step, wherein the HMO-containing solution, preferably after a concentration step is sterile filtered and/or subjected to endotoxin removal, preferably by filtration of the purified solution through a 3 kDa filter. Said optional step is preferably conducted before the drying step. The sterile filtration step does not affect the purity of the HMO-containing solution.

In a preferred embodiment, the drying step comprises spray-drying of the neutral or sialylated HMO-containing stream, preferably consists of spray-drying of the neutral or sialylated HMO-containing stream.

Preferably, spray-drying leads to solidified neutral or sialylated HMO having an amorphous structure, i.e. an amorphous powder is obtained.

In an embodiment, spray-drying is performed at a concentration of the neutral or sialylated HMO of 20-60% (w/v), preferably 30-50% (w/v), more preferably 35-45% (w/v), and an inlet temperature of 110-150° C., preferably 120-140° C., more preferably 125-135° C. and/or an outlet temperature of 60-80° C., preferably 65-70° C.

In some embodiment, the neutral or sialylated HMO-containing stream fed into the spray-dryer has a Brix value of from about 8 to about 75% Brix. In some embodiments, the Brix value is from about 30 to about 65% Brix. In some embodiments, the Brix value is from about 50 to about 60% Brix. In some embodiments, the feed into the spray-dryer is at a temperature of from about 2 to about 70° C. immediately before being dispersed into droplets in the spray-dryer. In some embodiments, the feed into the spray-dryer is at a temperature of from about 30 to about 60° C. immediately before being dispersed into droplets in the spray-dryer. In some embodiments, the feed into the spray-dryer is at a temperature of from about 2 to about 30° C. immediately before being dispersed into droplets in the spray-dryer. In some embodiments, the spray-drying uses air having an air inlet temperature of from 120 to 280° C. In some embodiments, the air inlet temperature is from 120 to 210° C. In some embodiments, the air inlet temperature is from about 130 to about 190° C. In some embodiments, the air inlet temperature is from about 135 to about 160° C. In some embodiments, the spray-drying uses air having an air outlet temperature of from about 80 to about 110° C. In some embodiments, the air outlet temperature is from about 100 to about 110° C. In some embodiments, the spray-drying is carried out at a temperature of from about 20 to about 90° C. In some embodiments, the spray-dryer is a co-current spray-dryer. In some embodiments, the spray-dryer is attached to an external fluid bed. In some embodiments, the spray-dryer comprises a rotary disk, a high-pressure nozzle, or a two-fluid nozzle. In some embodiments, the spray-dryer comprises an atomizer wheel. In some embodiments, spray-drying is the final purification step for the desired neutral or sialylated HMO.

Alternatively, the drying-solidification step comprises an indirect drying method. For the purposes of this specification, indirect dryers include those devices that do not utilize direct contact of the material to be dried with a heated process gas for drying, but instead rely on heat transfer either through walls of the dryer, e.g. through the shell walls in the case of a drum dryer, or alternately through the walls of hollow paddles of a paddle dryer, as they rotate through the solids while the heat transfer medium circulates in the hollow interior of the paddles. Other examples of indirect dryers include contact dryers and vacuum drum dryers.

Alternatively, the drying-solidification step comprises freeze-drying.

Alternatively, the drying-solidification step comprises crystallization (provided that the HMO is obtainable in crystalline form).

### Further Optional Method Steps

A) In the method according to the invention, the separated HMO-containing stream is purified by ultrafiltration using a membrane having a MWCO of 500 Da to 5 kDa, wherein the active (top) layer of the membrane is not a polyamide material (“tight” UF). This step is to separate high molecular weight components being present in the HMO-containing stream that have not yet been separated from the neutral or sialylated HMO in step I) or II) of the method according to the invention and that have a molecular weight higher than that of the neutral or sialylated HMO to be purified. Such high molecular weight components may be residual proteins and peptides, residual DNAs, RNAs and their fragments, lipids, residual endotoxins, higher oligosaccharides etc. The approach that this step is performed after the rough biomass has already been separated from the HMO-containing stream in step I) of the method according to the invention results in an overall improved separation and purification efficiency.

The material of the ultrafiltration membrane applied in this step can be a ceramic or made of a synthetic or natural polymer, e.g. polysulfone, polyvinylidene fluoride, polyacrylonitrile, polypropylene, cellulose, cellulose acetate or polylactic acid, except for polyamide. This step can be applied in dead-end or cross-flow mode.

Furthermore, for the membrane applied in this step, the HMO rejection factor is less than 90% under process conditions, which allows to pass at least part of the HMO into the permeate with optional diafiltration to ensure the accumulation of the HMO therein and retaining most of the higher molecular weight impurities in the retentate. Preferably the rejection factor is less than 70% or 50%. Examples of suitable membranes include Synder XT (1 kDa), Synder VT (3 kDa), Suez (GE) PT (5 kDa), Microdyn UH004 (4 kDa), Tami ceramic fine UF membranes (1 kDa, 3 kDa, 5 kDa).

The step may comprise more than one membrane filtration step using a membrane with a MWCO different than that applied before, as long as other parameters of the membrane fit as disclosed above, e.g. applying two membrane filtration separations.

The ultrafiltration permeate obtained after this step is an aqueous solution containing the neutral or sialylated human milk oligosaccharide also referred to as the HMO-containing stream, whereas the ultrafiltration retentate comprises the above mentioned high molecular weight components that are to be separated from the HMO-containing stream.

In a preferred embodiment, the yield of the desired neutral or sialylated HMO in the permeate after the ultrafiltration step performed is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In an embodiment, the purified solution obtained after this step contains the neutral or sialylated HMO at a purity of ≥50%, preferably ≥60%, more preferably ≥70% most preferably ≥80%.

The purification step by ultrafiltration using a membrane having a MWCO of 500 Da to 5 kDa, wherein the active (top) layer of the membrane is not a polyamide material can be performed after step I) but before step II), especially when step I) does not comprise the use of an ultrafiltration membrane having a MWCO of 500 Da to 5 kDa, wherein the active (top) layer of the membrane is not a polyamide material. In other embodiment, the “tight” UF step disclosed above can be performed directly after step II) on the adsorbent resin eluate containing HMOs, especially when step II) directly follows step I).

B) In method according to the invention, the neutral or sialylated HMO-containing stream can be purified by nanofiltration.

Nanofiltration (NF) can be used to remove low molecular weight molecules smaller than the desired neutral or sialylated HMOs, such as mono- and disaccharides, short peptides, small organic acids, water, and salts.

The product stream, i.e. the neutral or sialylated HMO-containing steam, is the NF retentate. The nanofiltration membrane thus has a MWCO or a pore size that ensures the retention of the neutral or sialylated human milk oligosaccharide of interest, i.e. the MWCO of the nanofiltration membrane is adjusted accordingly.

Typically, the pore size of the nanofiltration membrane is from 0.5 nm to 2 nm and/or from 150 dalton (Da) molecular weight cut-off (MWCO) to 3500 Da MWCO.

In an embodiment, the membranes are in the range of 150-300 Da MWCO, which are defined as “tight” NF membranes.

In a preferred embodiment, the membranes are above 300 Da MWCO, and preferably not higher than 3500 Da MWCO. In said embodiment, the membranes are considered “loose” NF membranes.

In another preferred embodiment, the “loose” nanofiltration membrane has a molecular weight cut-off (MWCO) of 500-3500 Da and the active (top) layer of the membrane is preferably composed of polyamide, more preferably piperazine-based polyamide. Thereby, the retention of tri- or higher oligosaccharides is ensured and at least part of the disaccharides is allowed to pass the membrane. In this embodiment, the applied nanofiltration membrane shall be tight for tri- and higher oligosaccharides for them to be efficiently retained. At the same time, the membrane shall be relatively loose for MgSO₄, that is its rejection is about 50-90%, in order that disaccharides can pass the membrane. This way, it is possible to separate e.g. lactose, which is a precursor in making human milk oligosaccharides e.g. by fermentation, from the neutral or sialylated human milk oligosaccharides product with a good efficacy, and additionally a substantial part of divalent ions also passes to the permeate. In some embodiments, the MgSO₄ rejection factor is 60-90%, 70-90%, 50-80%, 50-70%, 60-70% or 70-80%. Preferably, the MgSO₄ rejection factor on said membrane is 80-90%. Preferably, the membrane has a rejection factor for NaCl that is lower than that for MgSO₄. In one embodiment, the rejection factor for NaCl is not more than 50%. In another embodiment, the rejection factor for NaCl is not more than 40%. In another embodiment, the rejection factor for NaCl is not more than 30%. In this latter embodiment, a substantial reduction of all monovalent salts in the retentate is also achievable. In said embodiment, the membrane is a thin-film composite (TFC) membrane. An example of a suitable piperazine-based polyamide TFC membrane is TriSep® UA60. Other examples of suitable NF membranes include Synder NFG (600-800 Da), Synder NDX (500-700 Da), and TriSep® XN-45 (500 Da).

Preferably, the yield of the desired neutral or sialylated HMO in the retentate after a nanofiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In a preferred embodiment, the nanofiltration step comprises a diafiltration step, that is the nanofiltration step conducted in diafiltration mode. Preferably, the diafiltration step follows the aforementioned (conventionally conducted) nanofiltration step.

Diafiltration is a process that involves the addition of purified water to a solution during membrane filtration process in order to remove membrane permeable components more efficiently. Thus, diafiltration can be used to separate components on the basis of their properties, in particular molecular size, charge or polarity by using appropriate membranes, wherein one or more species are efficiently retained and other species are membrane permeable.

In a preferred embodiment, diafiltration and nanofiltration is combined within one step (referred to as nanofiltration/diafiltration or NF/DF) in which diafiltration is executed while using a nanofiltration membrane that is effective for the separation of low molecular weight compounds and/or salts from the neutral or sialylated HMOs. Diafiltration with “loose” NF membrane as defined above, is particularly efficient for both mono- and divalent salts removal and disaccharides removal from neutral or sialylated HMOs.

In a preferred embodiment, the DF step or the NF/DF step is performed so that the pH is set below 5.0, preferably, below 4.5, advantageously below 4.0, but preferably not less than 3.0. Under this condition, salts comprised of monovalent cations such as sodium salts (that is sodium ion together with the co-anion(s)) are effectively removed, giving rise to a low-salt or a practically salt-free purified solution containing a neutral or sialylated HMO in the retentate.

In an embodiment, a second nanofiltration step is carried out in the method according to the invention. In said second nanofiltration step, the nanofiltration membrane is either a “loose” NF membrane or a “tight” NF membrane. The second optional nanofiltration step is performed after the first nanofiltration step, but is preferably performed before step III) of the method according to the invention.

Likewise, a second diafiltration can be performed in the method according to the invention. This second optional diafiltration step can also be combined with the second nanofiltration step. This second NF/DF step, when “loose” NF membrane is applied as disclosed above, is performed so that the pH is set below 5.0, preferably below 4.5, advantageously below 4.0, but preferably not less than 3.0.

In an embodiment, the purified solution obtained after nanofiltration contains the neutral or sialylated HMO at a purity of ≥80%, preferably ≥85%, more preferably ≥90%.

In an embodiment, the purified solution obtained after nanofiltration is free proteins and/or recombinant genetic material.

Preferably, the membrane applied in the NF step is typically a “loose” NF membrane as disclosed above, that is it has a molecular weight cut-off (MWCO) of 500-3500 Da, the active (top) layer of the membrane is composed of polyamide, more preferably piperazine-based polyamide, the membrane has a MgSO₄ rejection factor of about 50-90% and preferably a NaCl rejection factor of not more than 50%, and the nanofiltration step is preferably performed so that the pH is set below 5.0, preferably below 4.5, advantageously below 4.0, but preferably not less than 3.0. The purification of the HMO-stream with “loose” NF membrane can be performed after step I) but before step II), especially when step I) does comprise the use of an ultrafiltration membrane. In other embodiment, the “loose” NF step disclosed above can be performed directly after step II) on the adsorbent resin eluate containing HMOs. In other embodiment, the “loose” NF step disclosed above can be performed directly after a “tight” UF step on the UF permeate, especially when the “tight” UF step disclosed above is performed directly after step II) on the adsorbent resin eluate containing HMOs, especially when step II) directly follows step I). In other embodiment, the “loose” NF step disclosed above can be performed directly after step II) on the adsorbent resin eluate containing HMOs.

C) In one embodiment, an electrodialysis (ED) step to demineralize the HMO-containing stream can be part of the method of the invention. The ED step is typically applied to substitute a nanofiltration step. So, the optional ED step preferably follows a “tight” UF purification step and operated on the UF permeate when the method does not contain a nanofiltration step.

D) In another embodiment, the method according to the invention including its preferred and more preferred realizations further comprises a step, wherein the purified neutral or sialylated HMO-containing solution, preferably after concentration and before the drying step comprised in step III), is sterile filtered and/or subjected to endotoxin removal, preferably by filtration of the purified solution through a 3 kDa filter or using a membrane having less than 0.5 μm, less than 0.4 μm, less than 0.3 μm, or less than 0.2 μm pore size. It is noted though that the sterile filtration step does not contribute to the solution of the technical problem, namely to purify HMOs from fermentation broth in which they have been produced and separate them from biomass, proteins, protein fragments, fragments of genetic material originated from genetically modified microorganism, salts, fermentation additives, metabolic by-products, especially non-carbohydrate by-products, colour bodies, etc., and thus to make them suitable for human administration. When the method disclosed above is carried out under suitably sterile conditions, sterile filtration is not necessary. Nevertheless, according to one embodiment, the sterile filtration step, disclosed above, may be part of the method of the invention.

### Particular Embodiments of the Invention

In a preferred embodiment, the method according to the present invention does not include an ion exchange resin treatment step, but at least a cation exchange resin treatment step.

In a preferred embodiment, the method according to the invention does not comprise a purification/decolourization step with active carbon.

In a further preferred embodiment, the method according to the invention comprises or consists of the following steps:
I. separating an HMO-containing stream from biomass,
II. purifying the HMO-containing stream with an adsorbent resin, and
III. concentrating and drying the purified HMO-containing stream to obtain a neutral or sialylated HMO in solidified form,
provided that the concentration step is optional when the drying step is freeze-drying,
and wherein the separated HMO-containing stream after step I) is purified by ultrafiltration using a membrane having a MWCO of 500 Da to 5 kDa, wherein the active (top) layer of the membrane is not a polyamide material, provided that said membrane was not used in step I).

In this regard, said preferred embodiment comprises or consists of the following steps, preferably in consecutive order:
separating an HMO-containing stream from biomass, provided that ultrafiltration using a membrane having a MWCO of 500 Da to 5 kDa, wherein the active (top) layer of the membrane is not a polyamide material, is not comprised,
purification of the separated HMO-containing stream by ultrafiltration using a membrane having a MWCO of 500 Da to 5 kDa, wherein the active (top) layer of the membrane is not a polyamide material,
purifying the ultrafiltration permeate with an adsorbent resin, and
concentrating and drying the adsorbent resin eluate containing HMO to obtain a neutral or sialylated HMO in solidified form,
provided that the concentration step is optional when the drying step is freeze-drying.

In another preferred embodiment, the method according to the invention comprises or consists of the following steps:
I. separating an HMO-containing stream from biomass,
II. purifying the HMO-containing stream with an adsorbent resin, and
III. concentrating and drying the purified HMO-containing stream to obtain a neutral or sialylated HMO in solidified form,
provided that the concentration step is optional when the drying step is freeze-drying,
and wherein the separated HMO-containing stream after step I) or the adsorbent resin eluate after step II) is purified by nanofiltration using a membrane having a molecular weight cut-off (MWCO) of 500-3500 Da, and the active (top) layer of the membrane is composed of polyamide.

In this regard, said preferred embodiment comprises or consists of the following steps, preferably in consecutive order:
separating an HMO-containing stream from biomass,
purification of the separated HMO-containing stream by nanofiltration using a membrane having a molecular weight cut-off (MWCO) of 500-3500 Da, and the active (top) layer of the membrane is composed of polyamide,
purifying the nanofiltration retentate with an adsorbent resin, and
concentrating and drying the adsorbent resin eluate containing HMO to obtain a neutral or sialylated HMO in solidified form,
or
separating an HMO-containing stream from biomass,
purifying the separated HMO-containing stream with an adsorbent resin,
purification of the adsorbent resin eluate by nanofiltration using a membrane having a molecular weight cut-off (MWCO) of 500-3500 Da, and the active (top) layer of the membrane is composed of polyamide, and
concentrating and drying the nanofiltration retentate containing HMO to obtain a neutral or sialylated HMO in solidified form,
provided that in any of the two cases above the concentration step is optional when the drying step is freeze-drying.

It is preferred that the membrane applied in the NF step has an active (top) layer of the membrane that is composed of piperazine-based polyamide, the membrane has a MgSO₄ rejection factor of about 50-90% and preferably a NaCl rejection factor of not more than 50%, and the nanofiltration step is preferably performed so that the pH is set below 5.0, preferably below 4.5, advantageously below 4.0, but preferably not less than 3.0. Further, it is preferred that the step “separating an HMO-containing stream from biomass” is an ultrafiltration step using a membrane having a MWCO of 500 Da to 5 kDa, wherein the active (top) layer of the membrane is not a polyamide material, preferably it is a ceramic membrane, when the NF step is the purification of the adsorbent resin eluate.

In another preferred embodiment, the method according to the invention comprises or consists of the following steps:
I. separating an HMO-containing stream from biomass,
II. purifying the HMO-containing stream with an adsorbent resin, and
III. concentrating and drying the purified HMO-containing stream to obtain a neutral or sialylated HMO in solidified form,
provided that the concentration step is optional when the drying step is freeze-drying, and wherein the adsorbent resin eluate after step II) is purified by ultrafiltration using a membrane having a MWCO of 500 Da to 5 kDa, wherein the active (top) layer of the membrane is not a polyamide material, provided that said membrane was not used in step I),
with the optional step of purifying the ultrafiltration permeate with either electrodialysis or by nanofiltration using a membrane having a molecular weight cut-off (MWCO) of 500-3500 Da, and the active (top) layer of the membrane is composed of polyamide.

In this regard, said preferred embodiment comprises or consists of the following steps, preferably in consecutive order:
separating an HMO-containing stream from biomass, provided that ultrafiltration using a membrane having a MWCO of 500 Da to 5 kDa, wherein the active (top) layer of the membrane is not a polyamide material, is not comprised,
purification of the separated HMO-containing stream with an adsorbent resin,
purifying the adsorbent resin eluate after by ultrafiltration using a membrane having a MWCO of 500 Da to 5 kDa, wherein the active (top) layer of the membrane is not a polyamide material,
optionally, purifying the ultrafiltration permeate with either electrodialysis or by nanofiltration using a membrane having a molecular weight cut-off (MWCO) of 500-3500 Da, and the active (top) layer of the membrane is composed of polyamide, and
concentrating and drying the obtained HMO-containing solution to provide a neutral or sialylated HMO in solidified form,
provided that in any of the two cases above the concentration step is optional when the drying step is freeze-drying.

It is preferred that the membrane applied in the optional NF step has an active (top) layer of the membrane that is composed of piperazine-based polyamide, the membrane has a MgSO₄ rejection factor of about 50-90% and preferably a NaCl rejection factor of not more than 50%, and the nanofiltration step is preferably performed so that the pH is set below 5.0, preferably below 4.5, advantageously below 4.0, but preferably not less than 3.0.

In the method according to the invention, including the preferred and more preferred embodiments disclosed above, it is further preferred that the adsorbent resin is an acid adsorbent derived from cross-linked polystyrene or polyacrylic polymers and partially functionalized with tertiary amine functional groups. More preferably, the adsorbent resin has a surface area of >400 m²/g, such as 400 to 1200 m²/g. Even more preferably, the adsorbent resin has a capacity of 0.1-0.3 eq/l.

### 3. Neutral or Sialylated Human Milk Oligosaccharide Produced by the Method According to the Invention

In another aspect, the invention relates to a neutral or sialylated human milk oligosaccharide obtained by the method according to the invention.

The neutral or sialylated HMO recovered and purified according to the method described in this specification can be amorphous or crystalline, preferably amorphous.

In a preferred embodiment, the purity of the neutral or sialylated HMO on a dry basis is greater than 80 wt. % for a single neutral or sialylated HMO based on dry matter; or for mixtures of HMOs, greater than 70% purity based on dry matter, for the combination. More preferably, the purity of a single neutral or sialylated HMO is greater than 90 wt. %.

In a preferred embodiment, the neutral or sialylated HMO has at least one of the following characteristics (by weight): <2% lactulose, <3% fucose, <1% galactose, or <3% glucose.

In an embodiment, the neutral or sialylated HMO has a fines fraction (less than or equal to 10 μm), of less than 10%, preferably less than 5%, more preferably less than 1%, most preferably less than 0.1%. The neutral or sialylated HMO also preferably has an average particle size (d50), of greater than 100 μm, more preferably greater than 150 μm, even more preferably greater than 200 μm.

The neutral or sialylated HMO produced by the method according to the invention, demonstrates good flowability. Preferably, the HMO has a Carr index of less than 30, where the Carr index (C) is determined by the formula C=100 (1−ρB/ρT), where ρB is the freely settled bulk density of the powder, and ρT is the tapped bulk density of the powder after “tapping down”. For free-flowing solids, the values bulk and tapped density would be similar, so the value is small. For poorer flowing solids, the differences between these values would be larger, so that the Carr index would be larger.

In a preferred embodiment, the neutral or sialylated HMO has a water content of less than 15 wt. %, less than 10 wt. %, less than 9 wt. %, less than 8 wt. %, less than 7 wt. %, or less than 6 wt. %. In order to optimize product recovery, preferably, the neutral or sialylated HMO has a pH greater than 3.0 in at least 5% solution. Typically, this is achieved by adjusting the pH of the HMO-containing stream to greater than 3.0 prior to the drying step. Preferably, the neutral or sialylated HMO has a pH of from 4 to 7, more preferably from 4.5 to 5.5.

Preferably, the HMO is a neutral HMO. In an embodiment, the neutral HMO is preferably selected from the group consisting of 2′-fucosyllactose, 3-fucosyllactose, 2′,3-difucosyllactose (DFL), lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-neofucopentaose V (alternative name: lacto-N-fucopentaose VI), lacto-N-difucohexaose I, lacto-N-difucohexaose II, lacto-N-difucohexaose III, 6′-galactosyllactose, 3′-galactosyllactose, lacto-N-hexaose, lacto-N-neohexaose, and any mixture thereof. More preferably, the HMO is 2′-fucosyllactose, 3-fucosyllactose, 2′,3-difucosyllactose, lacto-N-triose II, lacto-N-tetraose, lacto-N-neotetraose or a lacto-N-fucopentaose, more preferably 2′-fucosyllactose, 3-fucosyllactose or 2′,3-difucosyllactose.

In an embodiment, the sialylated HMO is selected from the group consisting of 3′-sialyllactose (3′-SL) and 6′-sialyllactose (6′-SL).

In an embodiment, the neutral or sialylated HMO obtained by the method according to the invention, is incorporated into a food product (e.g. human or pet food), dietary supplement or medicine product.

In some embodiments, the neutral or sialylated HMO is incorporated into a human baby food (e.g. infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to roughly mimic human breast milk. In some embodiments, a neutral or sialylated HMO purified by a method in this specification is included in infant formula to provide nutritional benefits similar to those provided by one or more neutral or sialylated HMOs in human breast milk. In some embodiments, the neutral or sialylated HMO is mixed with one or more ingredients of the infant formula. Examples of infant formula ingredients include skimmed milk, carbohydrate sources (e.g. lactose), protein sources (e.g. whey protein concentrate and casein), fat sources (e.g. vegetable oils-such as palm, high oleic safflower oil, rapeseed, coconut and/or sunflower oil; and fish oils), vitamins (such as vitamins A, B, B2, C and D), minerals (such as potassium citrate, calcium citrate, magnesium chloride, sodium chloride, sodium citrate and calcium phosphate).

Hence, another aspect of the invention relates to a neutral or sialylated human milk oligosaccharide obtained by the method according to the invention for use in medicine.

Hence, another aspect of the invention relates to the use of a neutral or sialylated human milk oligosaccharide obtained by the method according to the invention for food and/or feed applications.

Hence, another aspect of the invention relates to a food or cosmetic product comprising the neutral or sialylated human milk oligosaccharide obtained by the method according to the invention.

### EXAMPLES

### Example 1: Purification of DFL from Fermentation Broth

DFL-containing broth was generated by fermentation using a genetically modified E. coli strain of LacZ⁻, LacY⁺ phenotype, wherein said strain comprises recombinant genes encoding an α-1,2-fucosyltransferase and an α-1,3-fucosyltransferase enzyme which are able to transfer the fucosyl moiety of GDP-fucose to the internalized lactose and genes encoding a biosynthetic pathway to GDP-fucose. The fermentation was performed by culturing the strain in the presence of exogenously added lactose and a suitable carbon source, thereby producing DFL which was accompanied with 2′-FL, 3-FL and unreacted lactose as major carbohydrate impurities in the fermentation broth.

The obtained fermentation broth (13.97 kg) was acidified with H₂SO₄ to pH=4.0 then processed by ultrafiltration in an MMS SW18 membrane filtration system equipped with a 15 kDa ceramic membrane (Tami “INSIDE CéRAM” 1178×25 mm element with 39-channels, area 0.5 m²) at 50-60° C., trans membrane pressure (TMP) of 3 bar (cross flow: 600 l/h). When 6500 g of permeate was collected, continuous addition of DF water (total of 14 kg) was initiated at a 4.5 l/h of flow rate. In the end, 20169 g of UF permeate was collected and 6916 g of UF retentate was discarded. The UF permeate had the following parameters: Brix 4.0, conductivity 11.77 mS/cm, pH 4.66, protein: 1534 ppm, color index CI_400>100 (brown color).

A part of the obtained brown-colored UF permeate (2 l) was passed through a Dowex Optipore SD-2 resin (170 ml, water content of the dry resin: 58%, surface area: around 800 m²/g, dry weight capacity: 0.8 eq/kg) packed in a XK-16/100 column to give ca 2 l of nearly colorless solution. The resin regenerated with 2 BV (340 ml) of 2% HCl-solution, water, and then at 60° C. with 2 BV of 2% NaOH-solution, water, 2 BV of 2% NaCl-solution and water. The resin treatment was repeated in total of 3 times each with the new portion of UF permeate (total 3×2l). Before the last run, the resin was pre-treated with additional 2 BV of 2% HCl-solution followed by water. Color index CI_400 of the resin eluates was between 2.7-5.8, the protein content was 92-164 ppm.

The combined resin eluate (ca. 6 l) was processed by cross-flow loose nanofiltration with Trisep UA60 membrane (spiral-wound size 1812, area 0.23 m², MWCO of 1000-3500 Da) with diafiltration to give NF retentate 1 (1044 g, Brix 3.8, cond. 0.215 mS/cm, pH 6.1) and NF permeate 1 (23242 g, cond. 3.48 mS/cm, pH 6.1). The permeate 1 was re-processed by NF/DF to give NF retentate 2 (1036 g, Brix 3.2, cond. 1.05 mS/cm, pH 5.77) and NF permeate 2 (18750+23795 g, discarded). The combined NF retentate was concentrated and freeze-dried (purity by HPLC: DFL 82.2%, 2′-FL 14.0% %, 3-FL 0.9%, lactose 0.2%; protein: 68 ppm).

A part of the obtained solid was crystallized from aqueous ethanol to give DFL with >90% purity.

The resin treatment step not only removed the colour completely, but also reduced the protein concentration by an order of magnitude.

### Example 2: Determination of a Substance Rejection Factor on a Membrane

The NaCl and MgSO₄ rejection on a membrane is determined as follows: in a membrane filtration system, a NaCl (0.1%) or a MgSO₄ (0.2%) solution is circulated across the selected membrane sheet (for Tami: tubular module) while the permeate stream is circulated back into the feed tank. The system is equilibrated at 10 bars and 25° C. for 10 minutes before taking samples from the permeate and retentate. The rejection factor is calculated from the measured conductivity of the samples: (1−κₚ/κᵣ). 100, wherein κₚ is the conductivity of NaCl or MgSO₄ in the permeate and Kr is the conductivity of NaCl or MgSO₄ in the retentate.

| | | | NaCl rej. factor | | MgSO₄ rej. factor | |
|---|---|---|---|---|---|---|
| | | | supplier | lab. | supplier | lab. |
| membrane | active layer | MWCO | spec. | measurement | spec. | measurement |
| Trisep | piperazine- | 1000- | — | 10% | 80% | 81-89% |
| UA60 | PA | 3500 | | | | |
| Tami | ceramic | 1000 | — | — | — | 0% |

A carbohydrate rejection factor is determined in a similar way with the difference that the rejection factor is calculated from the concentration of the samples (determined by HPLC): (1−Cₚ/Cᵣ)·100, wherein Cₚ is the concentration of the carbohydrate in the permeate and Cᵣ is the concentration of the carbohydrate in the retentate.

### Example 3: Comparison of MgSO₄ and Na₂SO₄ Rejection

2.01 of 0.2% MgSO₄ solution were loaded into an MMS SW18 system equipped with 1812-size spiral wound Trisep UA60 element (piperazine-amide, MWCO 1000-3500 Da, membrane area 0.23 m²). The system was run at 400 l/h cross-flow with permeate circulating back to the feed tank. It was equilibrated for at least 5 min or until constant conductivity in the permeate under each condition. The pH was adjusted by adding a small amount of 25% H₂SO₄ solution. The conductivity of the permeate and the retentate are disclosed in the table below.

MgSO₄ rejection vs pH

| | | 25% | | | | | | |
|---|---|---|---|---|---|---|---|---|
| | | H₂SO₄ | | | | Retentate | Permeate | |
| TMP | T | added | pH | Flow | Flux | conductivity | conductivity | Rejection |
| (bar) | (° C.) | (μl) | (retentate) | (l/h) | (l/m²h) | (mS/cm) | (mS/cm) | factor |
| 10 | 24.7 | 0 | 6.06 | 21.1 | 91.6 | 2.100 | 0.785 | 62.61% |
| 10 | 25.2 | 20 | 4.91 | 20.9 | 90.8 | 2.100 | 0.587 | 72.05% |
| 10 | 24.9 | 40 | 4.35 | 20.9 | 90.8 | 2.130 | 0.624 | 70.70% |
| 10 | 25.1 | 80 | 3.93 | 20.5 | 89.2 | 2.170 | 0.520 | 76.04% |
| 10 | 25.1 | 160 | 3.56 | 20.2 | 87.7 | 2.260 | 0.538 | 76.19% |

The same experiment was performed with 0.2% Na₂SO₄ solution.

Na₂SO₄ rejection vs pH

| | | 25% | | | | | | |
|---|---|---|---|---|---|---|---|---|
| | | H₂SO₄ | | | | Retentate | Permeate | |
| TMP | T | added | pH | Flow | Flux | conductivity | conductivity | Rejection |
| (bar) | (° C.) | (μl) | (retentate) | (l/h) | (l/m²h) | (mS/cm) | (mS/cm) | factor |
| 10 | 22.0 | 0 | 5.70 | 16.7 | 72.6 | 2.840 | 0.0755 | 97.34% |
| 10 | 22.2 | 10 | 5.24 | 15.6 | 67.8 | 2.840 | 0.1706 | 93.99% |
| 10 | 22.0 | 30 | 4.39 | n/a | n/a | 2.840 | 0.824 | 70.99% |
| 10 | 22.2 | 70 | 3.91 | 13.6 | 59.1 | 2.850 | 1.706 | 40.14% |
| 10 | 22.2 | 110 | 3.71 | 18.0 | 78.3 | 2.880 | 1.939 | 32.67% |
| 10 | 22.0 | 190 | 3.48 | 18.4 | 80.0 | 2.970 | 2.090 | 28.67% |
| 10 | 22.0 | 350 | 3.21 | 18.4 | 80.0 | 3.060 | 2.230 | 27.12% |
| 10 | 22.0 | 670 | 2.94 | 18.4 | 80.0 | 3.280 | 2.500 | 23.78% |
| 10 | 22.0 | 1310 | 2.65 | 18.1 | 78.7 | 3.730 | 2.870 | 23.06% |

It was demonstrated that the sodium salt rejection with divalent counter-ion such as sulphate is strongly pH dependent in case of NF with polyamide membrane. Therefore, inorganic anions with sodium cation can be effectively removed in a NF/DF step when the DF is conducted at a pH of less than 4.5, advantageously less than 4.0, resulting in a practically salt-free solution (as assessed from conductivity). In this regard, no basic anionic resins are necessary to use to obtain a salt-free solution.

1. A method for the purification of a neutral or sialylated human milk oligosaccharide (HMO) from a fermentation broth, comprising the steps of:
I. separating an HMO-containing stream from biomass;
II. purifying the HMO-containing stream with an adsorbent resin, and
III. concentrating and drying the purified HMO-containing stream to obtain the neutral or sialylated HMOs in solidified form.

2. The method according to claim 1, the concentration step is optional when the drying step is freeze-drying.

3. The method according to claim 1, wherein the method does not comprise treatment with ion exchange resin.

4. The method according to claim 1, wherein the method does not comprise purification/decolourization step with active carbon.

5. The method according to claim 1, wherein the separated HMO-containing stream after step I) or the adsorbent resin eluate after step II) is purified by nanofiltration using a membrane having a molecular weight cut-off (MWCO) of 500-3500 Da, and the active (top) layer of the membrane is composed of polyamide.

6. The method according to claim 5, wherein step I) is ultrafiltration using a membrane having a MWCO of 500 Da to 5 kDa, wherein the active (top) layer of the membrane is not a polyamide material.

7. The method according to claim 5, wherein the nanofiltration membrane has an active layer of the membrane composed of piperazine-based polyamide, and its MgSO₄ rejection is about 50-90%.

8. The method according to claim 7, wherein the nanofiltration step is performed so that the pH is set below 5.0.

9. The method according to claim 1, wherein the concentration step before drying is evaporation or nanofiltration.

10. The method according to claim 1, wherein the drying step is spray-drying or freeze-drying.

11. The method according to claim 1, wherein the adsorbent resin is an acid adsorbent derived from cross-linked polystyrene or polyacrylic polymers and partially functionalized with tertiary amine functional groups.

12. The method according to claim 11, wherein the adsorbent resin has a surface area of >400 m²/g, preferably the adsorbent resin has an acid adsorbent capacity of 0.6-1.0 eq/kg on dry weight.

13. The method according to claim 1, wherein the HMO is a neutral HMO.

14. The method according to claim 13, wherein the neutral HMO is selected from the group consisting of: 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-fucopentaose VI, lacto-N-difucohexaose I, lacto-N-difucohexaose II, lacto-N-difucohexaose III, 6′-galactosyllactose, 3′-galactosyllactose, lacto-N-hexaose and lacto-N-neohexaose.

15. The method according to claim 1, wherein the HMO is a sialylated HMO.

16. The method according to claim 15, wherein the sialylated HMO is 3′-sialyllactose (3′-SL) or 6′-sialyllactose (6′-SL).

17. The method according to claim 13, wherein the neutral HMO is selected from the group consisting of: 2′-fucosyllactose, 3-fucosyllactose, 2′,3-difucosyllactose, lacto-N-triose II, lacto-N-tetraose, lacto-N-neotetraose and a lacto-N-fucopentaose.

18. The method according to claim 13, wherein the neutral HMO is selected from the group consisting of: 2′-fucosyllactose, 3-fucosyllactose and 2′,3-difucosyllactose.

19. The method according to claim 8, wherein the pH is below 4.5.

20. The method according to claim 19, wherein the pH ranges from 3.0 to 4.5.
